(19) United States
(12) Patent Application Publication
(10) Pub. No.: US 2025/0363625 A1
(43) Pub. Date: Nov. 27, 2025

(54) VIRAL PARTICLE DETECTION METHOD, INFORMATION PROCESSING DEVICE, AND VIRAL PARTICLE DETECTION PROGRAM

(71) Applicant: The University of Tokyo, Tokyo (JP)

(72) Inventors: Takashi OKADA, Tokyo (JP); Yasunari MATSUZAKA, Tokyo (JP); Mikako WADA, Tokyo (JP)

(21) Appl. No.: 18/871,646

(22) PCT Filed: May 31, 2023
(86) PCT No.: PCT/JP2023/020378

§ 371 Date: Dec. 4, 2024

Related U.S. Application Data
(60) Provisional application No. 63/349,783, filed on Jun. 7, 2022.

(51) Int. Cl. G06T 7/00; C12N 7/00; G01N 1/30; G06T 7/13; G06V 10/764; G06V 10/774; G06V 20/69

(52) U.S. Cl. CPC G06T 7/0012; C12N 7/00; G01N 1/30; G06T 7/13; G06V 10/764; G06V 10/774; G06V 20/698; C12N 2750/14111; G01N 2001/302; G06T 2207/10061; G06T 2207/20081; G06T 2207/30024; G06V 2201/03

(57) ABSTRACT

A viral particle detection method for detecting a type of a viral particle that is a test subject, includes: generating a plurality of pieces of training image data in which the viral particle appears; receiving, for each of the generated plurality of pieces of training image data, input of a combination of a type of the viral particle appearing in the training image data and position information of the viral particle in the training image data; generating a plurality of pieces of training data by associating the combinations input for each of the plurality of pieces of training image data with the respective pieces of training image data; and generating a trained model by performing machine learning on the generated plurality of pieces of training data.

### REFERENCE TO RELATED APPLICATIONS

This application claims the benefit of priority from earlier U.S. 63/349,783 (filed Jun. 7, 2022), the entire disclosure of which is incorporated herein by reference.

### TECHNICAL FIELD

The present invention relates to a viral particle detection method, an information processing device, and a viral particle detection program.

### BACKGROUND ART

For example, adeno-associated viruses (hereinafter also referred to as AAVs) that use primates such as humans as hosts are non-pathogenic viruses that can infect proliferating and non-proliferating cells. Also, AAV vectors, which are recombinant vectors of AAVs, are used as safe and effective gene therapy vectors for the treatment of various diseases due to their high efficiency in transmitting target genes carried on the viral genome (see Patent Literature 1).

### CITATION LIST

### Patent Literature

Patent Literature 1: Japanese Translation of PCT Application No. 2020-513230

### SUMMARY OF INVENTION

### Problems to be Solved by the Invention

Here, various viral vectors (hereinafter simply referred to as viral vectors), including the AAV vectors described above, may require further improvement in terms of safety and efficacy due to the presence of neutralizing antibodies, incomplete organ tropism, and the like.

For this reason, there is demand for a method according to which it is possible to, in the process of creating viral vectors, accurately detect (classify) each of the following: whole particles that encapsulate full-length DNA (DeoxyriboNucleic Acid) inside a capsid, hollow particles that are empty shells that do not contain DNA, damaged particles in which part of the particle is damaged, intermediates that encapsulate fragmented DNA, and the like.

In view of this, an object of the present invention is to provide a viral particle detection method, an information processing device, and a viral particle detection program according to which it is possible to detect viral particles with high accuracy.

### Means for Solving the Problems

According to an aspect of the embodiments, a viral particle detection method for detecting a type of a viral particle that is a test subject, comprising: generating a plurality of pieces of training image data in which the viral particle appears; receiving, for each of the generated plurality of pieces of training image data, input of a combination of a type of the viral particle appearing in the training image data and position information of the viral particle in the training image data; generating a plurality of pieces of training data by associating the combinations input for each of the plurality of pieces of training image data with the respective pieces of training image data; generating a trained model by performing machine learning on the generated plurality of pieces of training data; inputting a determination image data in which the viral particle appears into the trained model; acquiring an estimation result output from the trained model accompanying input of the determination image data; outputting the acquired estimation result as the combination corresponding to the viral particle appearing in the determination image data.

### ADVANTAGEOUS EFFECTS OF THE INVENTION

According to the viral particle detection method, information processing device, and viral particle detection program of the present invention, it is possible to detect viral particles with high accuracy.

### BRIEF DESCRIPTION OF DRAWINGS

FIG. 1 is a diagram showing an example of a configuration of the information processing system 10 according to the first embodiment.

FIG. 2 is a diagram showing an example of a configuration of the information processing device 1 according to the first embodiment.

FIG. 3 is a diagram illustrating an overview of the first embodiment.

FIG. 4 is a diagram illustrating an overview of the first embodiment.

FIG. 5 is a diagram illustrating an overview of the first embodiment.

FIG. 6 is a flowchart illustrating the image acquisition processing.

FIG. 7 is a flowchart illustrating the information acquisition processing.

FIG. 8 is a flowchart illustrating the main processing of the training processing.

FIG. 9 is a diagram illustrating a specific example of the processing of step S23.

FIG. 10 is a diagram illustrating a specific example of the processing of step S23.

FIG. 11 is a diagram illustrating a specific example of the processing of step S23.

FIG. 12 is a diagram illustrating a specific example of the training data DT4.

FIGS. 13A to 13D are diagrams illustrating the training processing in the first embodiment.

FIG. 14 is a diagram illustrating the training processing in the first embodiment.

FIG. 15 is a flowchart illustrating the estimation processing according to the first embodiment.

FIG. 16 is a diagram illustrating the estimation processing in the first embodiment.

### DESCRIPTION OF EMBODIMENTS

Hereinafter, embodiments of the present disclosure will be described with reference to the drawings. However, the description is not to be construed in a limiting sense and is not intended to limit the subject matter recited in the claims. In addition, various changes, substitutions, and alterations can be made without departing from the spirit and scope of the present disclosure. In addition, different embodiments can be combined with each other as appropriate.

### Example of Configuration of Information Processing System 10 According to First Embodiment

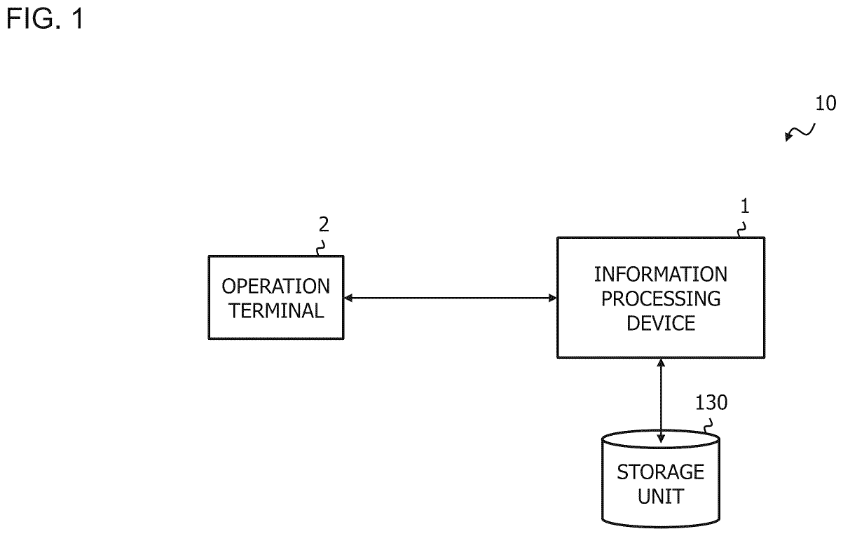

First, an example of a configuration of an information processing system 10 according to the first embodiment will be described. FIG. 1 is a diagram showing an example of a configuration of the information processing system 10 according to the first embodiment.

The information processing system 10 includes, for example, an information processing device 1, an operation terminal 2, and a storage unit 130. Note that the storage unit 130 may be a storage unit disposed outside the information processing device 1, or may be a storage unit mounted within the information processing device 1.

The operation terminal 2 is, for example, a mobile terminal such as a PC (Personal Computer) or a smartphone, and is a terminal through which an operator of the information processing device 1 (hereinafter also simply referred to as an operator) inputs necessary information and the like.

The information processing device 1 is, for example, a physical machine or a virtual machine, and performs processing for generating a trained model for detecting viral particles (hereinafter also simply referred to as training processing) and processing for detecting viral particles by using the trained model (hereinafter also simply referred to as estimation processing). Hereinafter, a case where the viral particles are viral vector particles will be described. Note that the trained model generated in the training processing may be, for example, a trained model based on YOLO (You Only Look Once).

Specifically, in the training processing, the information processing device 1 generates, for example, a plurality of pieces of image data (hereinafter also referred to as training image data) in which test subject viral vector particles (e.g., viral vector particles having capsids) appear. The test subject viral vector particles may be parvoviral vector particles such as, for example, dependoparvovirus vector particles including AAV viral vector particles, bocaparvovirus vector particles including AAV viral vector particles, erythroparvovirus vector particles including AAV viral vector particles, and protoparvovirus vector particles including AAV viral vector particles. The test subject viral vector particles may also be, for example, adenoviral vector particles including mastadenovirus vector particles. In addition, the test subject viral vector particles may be, for example, simplex viral vector particles. The test subject viral vector particles may also be, for example, beta-baculovirus vector particles. The test subject viral vector particles may also be, for example, inovirus vector particles. The test subject viral vector particles may also be, for example, Tequatrovirus vector particles. Then, for each of the generated plurality of pieces of image data, the information processing device 1 receives input of a combination of the type of the viral vector particle appearing in the image data and the position information of the viral vector particle in the image data (hereinafter simply referred to as a combination), for example. There are a plurality of types of viral vector particles including, for example, whole particles, hollow particles, damaged particles, and intermediates. Next, for each of the plurality of pieces of image data, the information processing device 1 generates a plurality of pieces of training data by, for example, associating the input combination with the image data. Thereafter, the information processing device 1 generates a trained model by, for example, performing machine learning on the generated plurality of pieces of training data.

In addition, in the estimation processing, the information processing device 1 inputs, for example, image data in which a viral vector particle appears (hereinafter also referred to as determination image data) into the trained model. Then, the information processing device 1 acquires, for example, an estimation result output from the trained model accompanying the input of the image data. Thereafter, the information processing device 1 outputs, for example, the acquired estimation result as a combination corresponding to the viral vector particle in the input image data.

That is, the information processing device 1 according to this embodiment generates a trained model by using a plurality of pieces of training data including, for example, image data, types of viral vector particles, and also position information of the viral vector particles in the image data.

This enables the information processing device 1 in this embodiment to detect, for example, each viral vector particle without manual intervention. Also, the information processing device 1 can, for example, detect each viral vector particle accurately and quickly.

Although the following will describe a case where the information processing system 10 includes one information processing device 1, the information processing system 10 may also include, for example, a plurality of information processing devices 1. Also, although the following will describe a case where the information processing system 10 has one operation terminal 2, the information processing system 10 may have, for example, a plurality of operation terminals 2.

Furthermore, the information processing device 1 and the operation terminal 2 may be, for example, a single device. Specifically, the information processing system 10 may not include the operation terminal 2 if, for example, a PC or a mobile terminal into which the operator can directly input information is used as the information processing device 1.

### Example of Configuration of Information Processing Device 1 of First Embodiment

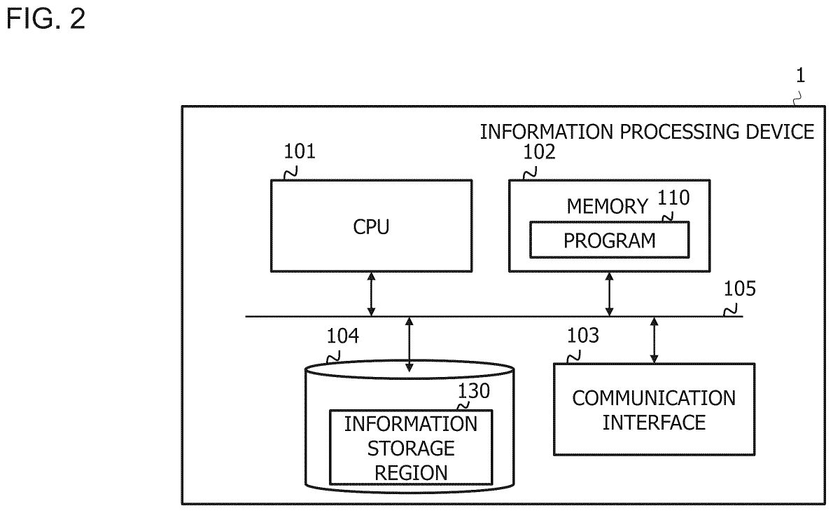

Next, an example of a configuration of the information processing device 1 will be described. FIG. 2 is a diagram showing an example of a configuration of the information processing device 1 according to the first embodiment.

As shown in FIG. 2, the information processing device 1 includes, for example, a CPU 101 that is a processor, a memory 102, a communication interface 103, and a storage medium 104. The units are connected to each other via a bus 105.

The storage medium 104 has a program storage area (not shown) that stores a program 110 for performing, for example, training processing and estimation processing (hereinafter, these are also collectively referred to as training processing and the like).

Also, the storage medium 104 has a storage unit 130 (hereinafter also referred to as an information storage region 130) that stores information to be used when performing, for example, training processing or the like. Note that the storage medium 104 may be, for example, an HDD (hard disk drive) or an SSD (solid state drive).

The CPU 101 executes, for example, a program 110 loaded from the storage medium 104 to the memory 102 to perform training processing and the like.

Furthermore, the communication interface 103 communicates with, for example, the operation terminal 2.

### Overview of First Embodiment

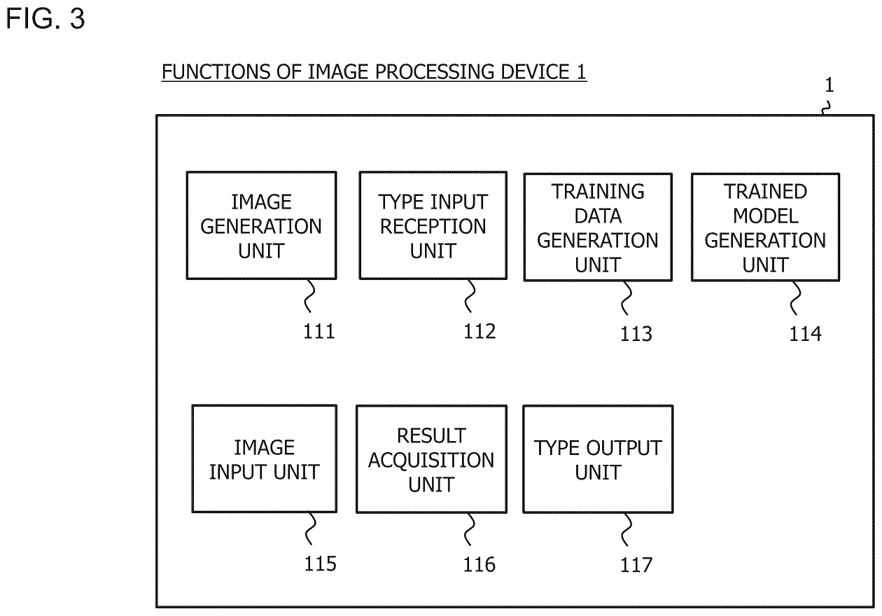

Next, an overview of the first embodiment will be described. FIGS. 3 to 5 are diagrams illustrating an overview of the first embodiment.

As shown in FIG. 3, the information processing device 1 realizes various functions including, for example, an image generation unit 111, a type input reception unit 112, a training data generation unit 113, a trained model generation unit 114, an image input unit 115, a result acquisition unit 116, and a type output unit 117.

Specifically, each of the image generation unit 111, the type input reception unit 112, the training data generation unit 113, and the trained model generation unit 114 is a function that realizes, for example, training processing. Also, each of the image generation unit 111, the image input unit 115, the result acquisition unit 116, and the type output unit 117 is a function that realizes, for example, estimation processing.

Note that in the following, a case will be described in which the training processing and the estimation processing are executed in the information processing device 1, but there is no limitation to this example. Specifically, either the training processing or the estimation processing may be executed in, for example, another information processing device (not shown) different from the information processing device 1.

First, the functions in the training processing will be described.

The image generation unit 111 generates, for example, a plurality of pieces of image data DT1 (training image data DT1) in which viral vector particles appear. Specifically, the image generation unit 111 controls, for example, a transmission electron microscope (hereinafter also referred to as a TEM) to capture images of viral vector particles and generate a plurality of pieces of image data DT1. Note that the image generation unit 111 may simply acquire, from the TEM, a plurality of pieces of image data DT1 captured by the TEM.

For each of the plurality of pieces of image data DT1 generated by the image generation unit 111, the type input reception unit 112 receives, for example, input of a combination of type information DT2 indicating the type of the viral vector particle appearing in the image data DT1 and position information DT3 indicating the position of the viral vector particle in the image data DT1.

Specifically, the type input reception unit 112 sequentially outputs, for example, the image data DT1 generated (acquired) by the image generation unit 111 to the operation terminal 2. Then, for example, the operator views the image data DT1 output to the operation terminal 2, and inputs a combination of the type information DT2 and the position information DT3 corresponding to the viral vector particle appearing in the image data DT1. Thereafter, the type input reception unit 112 receives, for example, the combination of the type information DT2 and the position information DT3 input by the operator. Note that if a plurality of viral vector particles appear in the image data DT1, the operator may input, for example, a combination of type information DT2 and position information DT3 corresponding to each of the plurality of viral vector particles.

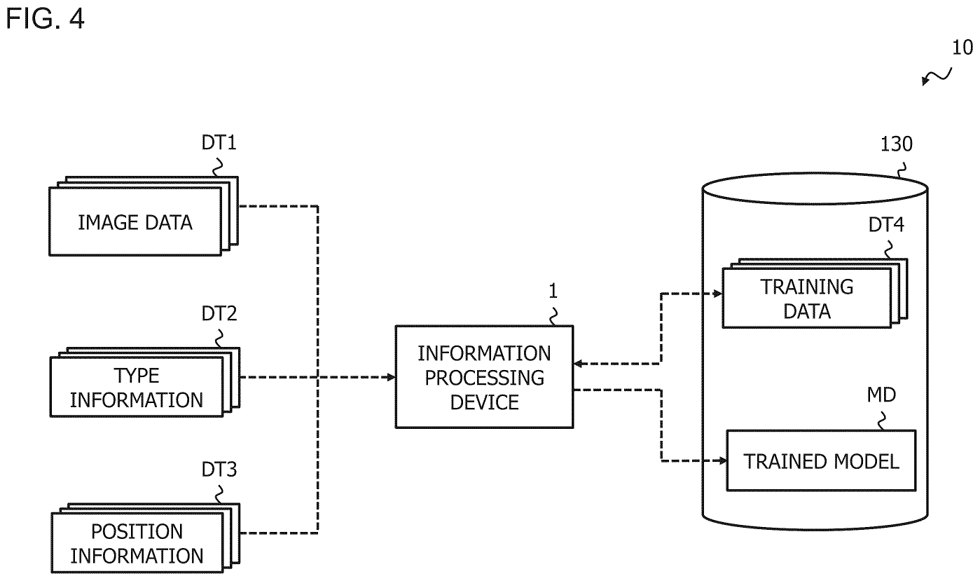

As shown in FIG. 4, the training data generation unit 113 generates a plurality of pieces of training data DT4 by, for example, associating combinations of the type information DT2 and the position information DT3 received by the type input reception unit 112 with the respective plurality of pieces of image data DT1 generated by the image generation unit 111. Then, the training data generation unit 113 stores the generated plurality of pieces of training data DT4 in the information storage region 130, for example.

As shown in FIG. 4, the trained model generation unit 114 generates a trained model MD, for example, by performing machine learning on the plurality of pieces of training data DT4 generated by the training data generation unit 113 (the plurality of pieces of training data DT stored in the information storage region 130). Then, the trained model generation unit 114 stores the generated trained model MD in the information storage region 130, for example.

Next, the functions in the estimation processing will be described.

The image generation unit 111 generates image data DT11 (determination image data DT11) in which viral vector particles appear, for example, in the same manner as in the training processing.

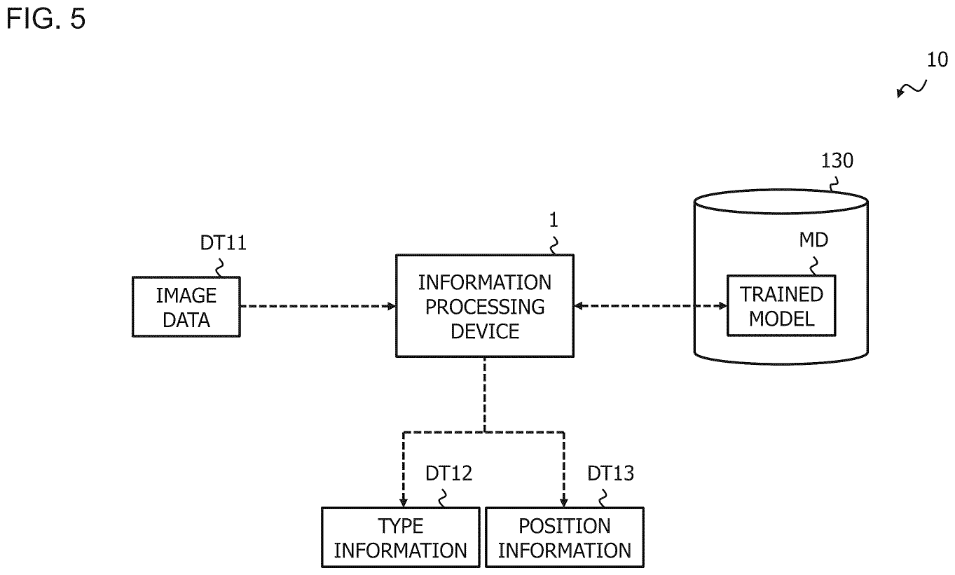

As shown in FIG. 5, the image input unit 115 inputs, for example, the image data DT11 generated by the image generation unit 111 to the trained model MD.

As shown in FIG. 5, the result acquisition unit 116 acquires, for example, an estimation result output from the trained model MD accompanying input of the image data DT11 by the image input unit 115. The estimation result is, for example, an estimation result for the combination of the type information DT12 and the position information DT13 corresponding to the viral vector particle appearing in the image data DT11. Note that if a plurality of viral vector particles appear in the image data DT11, the trained model MD may, for example, estimate a combination of type information DT12 and position information DT13 corresponding to each of the plurality of viral vector particles.

As shown in FIG. 5, for example, the type output unit 117 outputs the estimation result acquired by the result acquisition unit 116 as the combination of the type information DT12 and the position information DT13 corresponding to the viral vector particle appearing in the image data DT11 acquired by the image generation unit 111.

This enables the information processing device 1 in this embodiment to detect, for example, each viral vector particle without manual intervention. Also, the information processing device 1 can, for example, detect each viral vector particle with high accuracy.

### Flowchart of Training Processing in First Embodiment

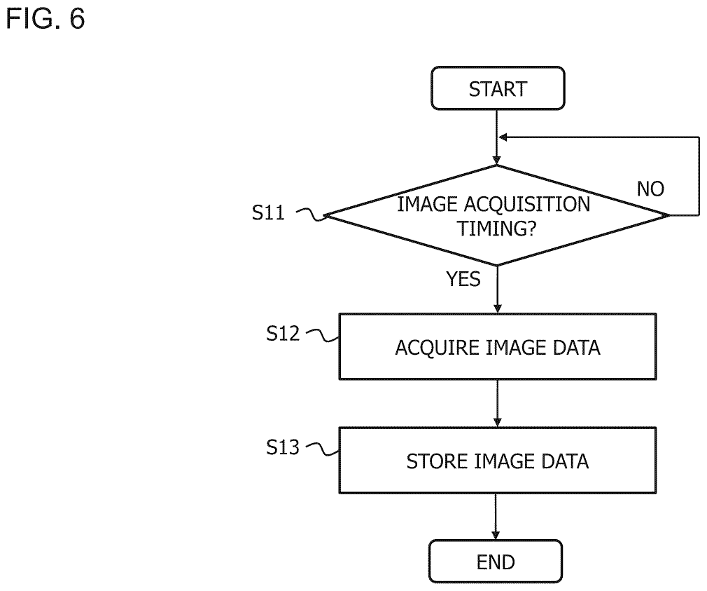

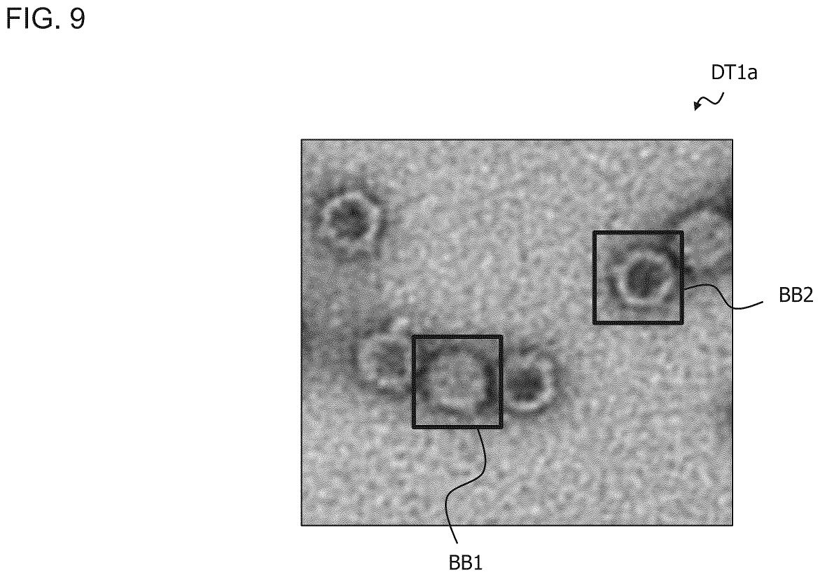

Next, the training processing in the first embodiment will be described. FIGS. 6 to 8 are flowcharts illustrating the training processing in the first embodiment. Also, FIGS. 9 to 14 are diagrams illustrating the training processing in the first embodiment.

First, the processing for acquiring the image data DT1 (hereinafter also referred to as image acquisition processing) in the training processing will be described. FIG. 6 is a flowchart illustrating the image acquisition processing.

As shown in FIG. 6, the image generation unit 111 waits until an image acquisition timing is reached, for example (NO in step S11). The image acquisition timing may be, for example, a timing designated by the operator. Specifically, the image acquisition timing may be, for example, a timing when the image data DT1 is captured by the TEM.

Then, if the image acquisition timing has been reached (YES in step S11), the image generation unit 111 acquires image data DT1 in which, for example, a viral vector particle appears (step S12). Specifically, the image generation unit 111 sequentially acquires, for example, a plurality of pieces of image data DT1 captured by the TEM from the TEM.

Thereafter, the image generation unit 111 stores, for example, the image data DT1 acquired in the processing of step S12 in the information storage region 130 (step S13).

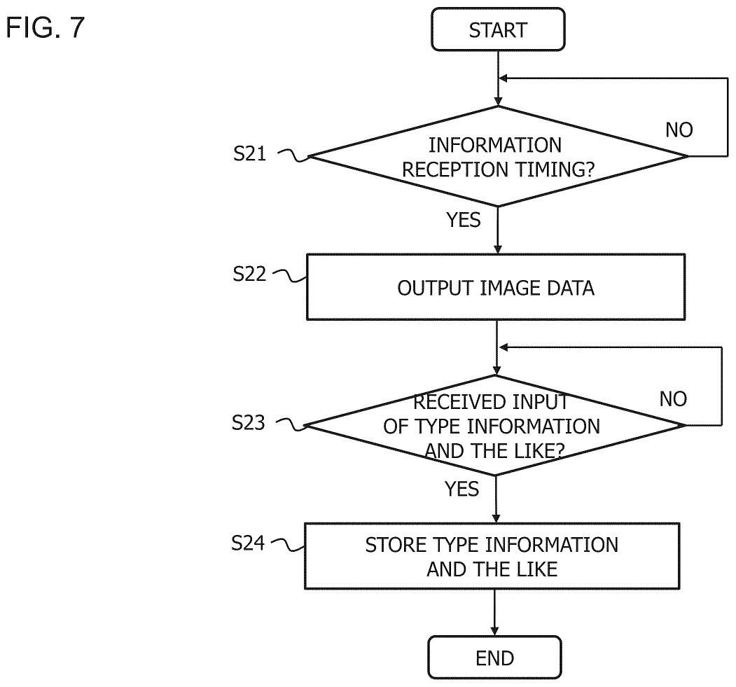

Next, the processing for acquiring the type information DT2 and the position information DT3 (hereinafter also referred to as information acquisition processing) in the training processing will be described. FIG. 7 is a flowchart illustrating the information acquisition processing.

As shown in FIG. 7, the type input reception unit 112 waits until an information acquisition timing is reached, for example (NO in step S21). The information acquisition timing may be, for example, a timing designated by the operator. Specifically, the information acquisition timing may be, for example, a timing immediately after the processing of step S13 is performed.

Then, when the information acquisition timing is reached (YES in step S21), the type input reception unit 112 outputs, for example, the image data DT1 acquired in the processing of step S12 to the operation terminal 2 (step S22).

Thereafter, the type input reception unit 112 waits, for example, until the combination of the type information DT2 of the viral vector particle appearing in the image data DT1 acquired in the processing of step S12 and the position information DT3 of the viral vector particle in the image data DT1 is input (NO in step S23). That is, the type input reception unit 112 waits until the operator inputs the combination of the type information DT2 and the position information DT3 via the operation terminal 2, for example.

Then, when the combination of the type information DT2 of the viral vector particle appearing in the image data DT1 acquired in the processing of step S12 and the position information DT3 of the viral vector particle in the image data DT1 is input (YES in step S23), the type input reception unit 112 stores, for example, the received combination of the type information DT2 and the position information DT3 in the information storage region 130 (step S24). That is, the type input reception unit 112 stores, in the information storage region 130, for example, the combination of the type information DT2 and the position information DT3 corresponding to each of the plurality of pieces of image data DT1 captured by the TEM. Hereinafter, a specific example of the processing of step S23 will be described.

### Specific Example of Processing of Step S23

FIGS. 9 to 11 are diagrams illustrating a specific example of the processing of step S23.

As shown in FIG. 9, for example, when image data DT1a is displayed on the operation terminal 2 in the processing of step S22, the operator inputs, on an output screen (not shown) of the operation terminal 2, a bounding box BB1 including a viral vector particle that can be determined to be a whole particle, and inputs type information DT2 indicating that the viral vector particle included in the bounding box BB1 is a whole particle. In this case, the type input reception unit 112, for example, receives the input type information DT2 (type information DT2 indicating a whole particle) and receives the coordinates of each vertex of the input bounding box BB1 as position information DT3 (position information DT3 of the whole particle).

Also, as shown in FIG. 9, for example, when the image data DT1a is displayed on the operation terminal 2 in the processing of step S22, the operator inputs, on the output screen of the operation terminal 2, a bounding box BB2 including a viral vector particle that can be determined to be a hollow particle, and inputs type information DT2 indicating that the viral vector particle included in the bounding box BB2 is a hollow particle. In this case, the type input reception unit 112, for example, receives the input type information DT2 (type information DT2 indicating a hollow particle) and receives the coordinates of each vertex of the input bounding box BB2 as position information DT3 (position information DT3 of the hollow particle).

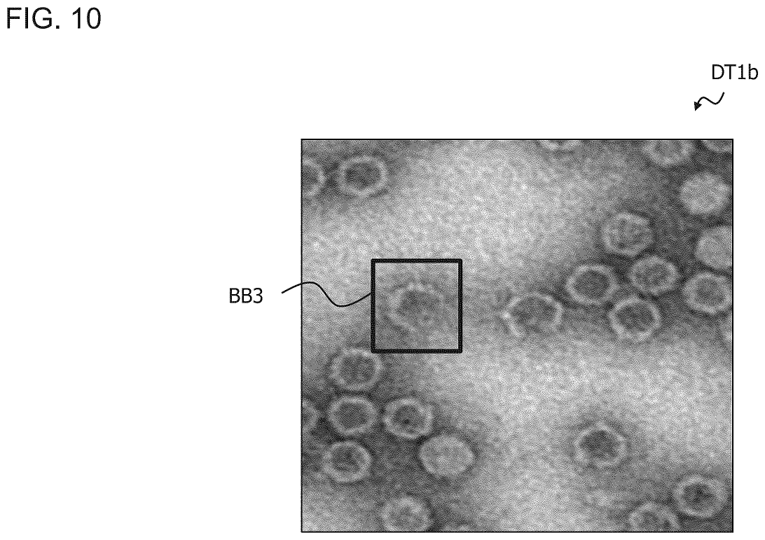

Also, as shown in FIG. 10, for example, when image data DT1b is displayed on the operation terminal 2 in the processing of step S22, the operator inputs, on the output screen of the operation terminal 2, a bounding box BB3 including a viral vector particle that can be determined to be a damaged particle, and inputs type information DT2 indicating that the viral vector particle included in the bounding box BB3 is a damaged particle. In this case, the type input reception unit 112, for example, receives the input type information DT2 (type information DT2 indicating a damaged particle) and receives the coordinates of each vertex of the input bounding box BB3 as position information DT3 (position information DT3 of the damaged particle).

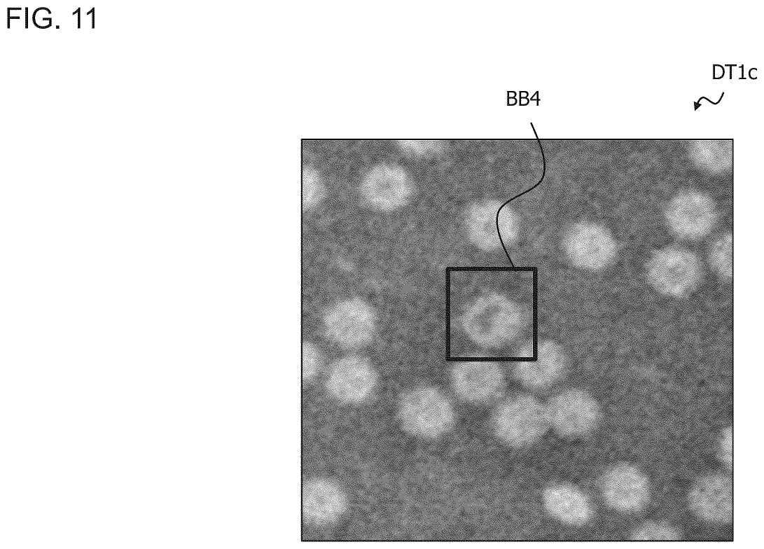

Also, as shown in FIG. 11, for example, when image data DT1c is displayed on the operation terminal 2 in the processing of step S22, the operator inputs, on the output screen of the operation terminal 2, a bounding box BB4 including a viral vector particle that can be determined to be an intermediate, and inputs type information DT2 indicating that the viral vector particle included in the bounding box BB4 is an intermediate. In this case, the type input reception unit 112, for example, acquires the input type information DT2 (type information DT2 indicating an intermediate) and acquires the coordinates of each vertex of the input bounding box BB4 as position information DT3 (position information DT3 of the intermediate).

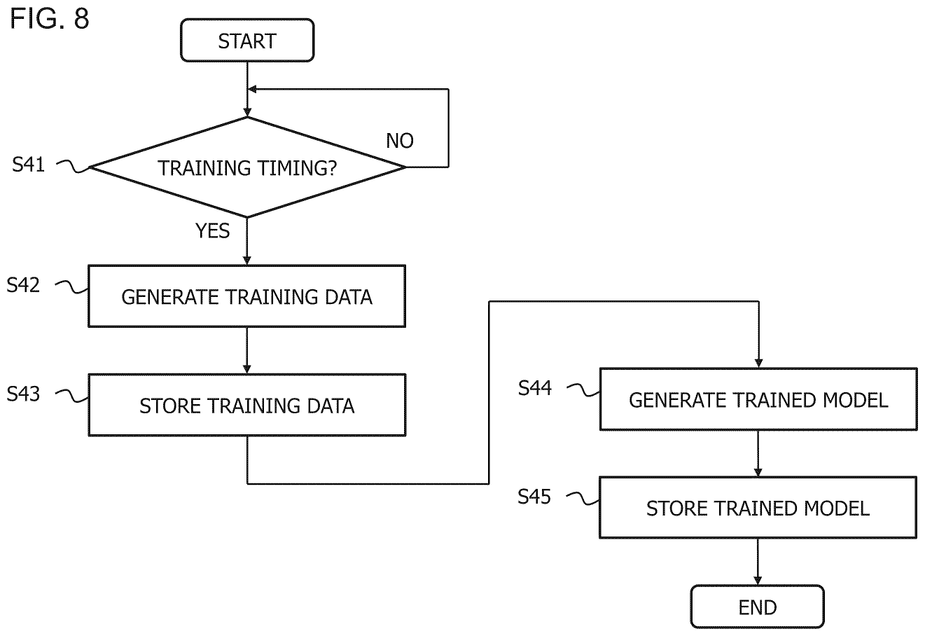

Next, the main processing of the training processing will be described. FIG. 8 is a flowchart illustrating the main processing of the training processing.

As shown in FIG. 8, the training data generation unit 113 waits until the training timing is reached, for example (NO in step S41). The training timing may be, for example, a timing designated by the operator. Specifically, the training timing may be, for example, a timing when the number of combinations of image data DT1, type information DT2, and position information DT3 stored in the information storage region 130 reaches the number (a predetermined number) of pieces of training data DT4 used to generate the trained model MD.

Then, when the training timing is reached (YES in step S41), for each of the plurality of pieces of image data DT1 acquired in the processing of step S12 (for each of the plurality of pieces of image data DT1 stored in the image storage region 130), for example, the training data generation unit 113 generates a plurality of pieces of training data DT4 (step S42) by associating the combination of the type information DT2 and the position information DT3 input in the processing of step S23 with the image data DT1.

Thereafter, the training data generation unit 113 stores, for example, the generated plurality of pieces of training data DT4 in the information storage region 130 (step S43). Hereinafter, a specific example of the plurality of pieces of training data DT4 will be described.

### Specific Example of Training Data DT4

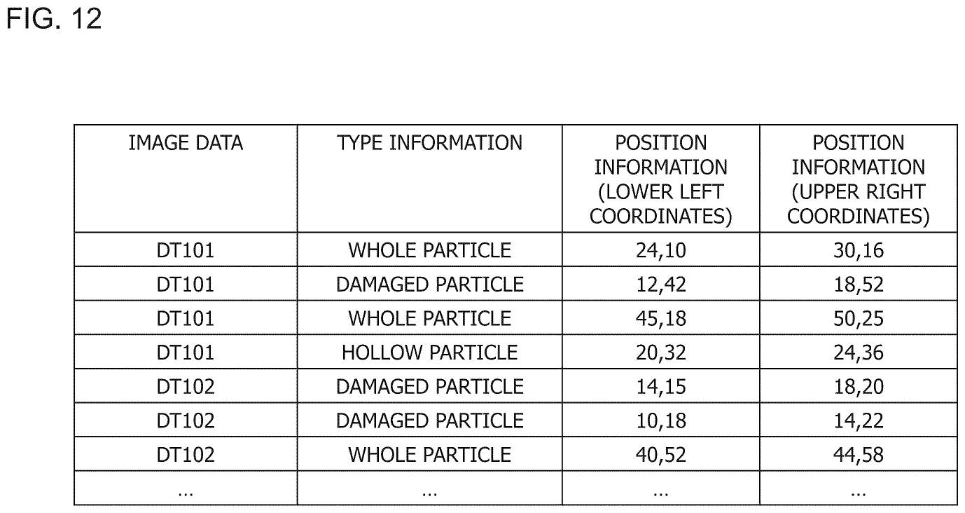

FIG. 12 is a diagram illustrating a specific example of the training data DT4.

The training data DT4 shown in FIG. 12 has, for example, the items “image data”, in which identification information of the image data DT1 acquired in the processing of step S12 is set, and “type information”, in which type information DT2 input in the processing of step S23 is set. The “type information” is set to, for example, “whole particle” indicating a type corresponding to a whole particle, “hollow particle” indicating a type corresponding to a hollow particle, “damaged particle” indicating a type corresponding to a damaged particle, or “intermediate” indicating a type corresponding to an intermediate.

In addition, the training data DT4 shown in FIG. 12 has the items “position information (lower left coordinates)” and “position information (upper right coordinates)”, in which the position information DT3 corresponding to the bounding box input in the processing of step S23 is set. In the “position information (lower left coordinates)”, for example, the lower left X coordinate and Y coordinate of the bounding box input in the processing of step S23 are set. In addition, in the “position information (upper right coordinates)”, for example, the upper right X and Y coordinates of the bounding box input in the processing of step S23 are set. Note that the training data DT4 may also include, for example, other items corresponding to the position information DT3 (e.g., upper left coordinates and lower right coordinates of the bounding box input in the processing of step S23).

Specifically, in the training data DT4 shown in FIG. 12, for example, in the first row of data (first training data DT4), “DT101” is set as the “image data”, “whole particle” is set as the “type information”, “24,10” is set as the “position information (lower left coordinates)”, and “30,16” is set as the “position information (upper right coordinates)”.

Also, in the training data DT4 shown in FIG. 12, for example, in the data in the second row (second training data DT4), “DT101” is set as the “image data”, “damaged particle” is set as the “type information”, “12,42” is set as the “position information (lower left coordinates)”, and “18,52” is set as the “position information (upper right coordinates)”.

Also, in the training data DT4 shown in FIG. 12, for example, in the data in the fifth row (fifth training data DT4), “DT102” is set as the “image data”, “damaged particle” is set as the “type information”, “14,15” is set as the “position information (lower left coordinates)”, and “18,20” is set as the “position information (upper right coordinates)”. Description of other data included in FIG. 12 will be omitted.

Note that the “position information (lower left coordinates)” and “position information (upper right coordinates)” in the training data DT4 may be set to normalized coordinates between 0 and 1 as the X coordinates and the Y coordinates.

Returning to FIG. 8, the trained model generation unit 114 generates a trained model MD by, for example, performing machine learning on the plurality of pieces of training data DT4 generated in the processing of step S42 (step S44).

Then, the trained model generation unit 114 stores, for example, the trained model MD generated in the processing of step S44 in the information storage region 130 (step S45).

This enables the information processing device 1 to generate a trained model MD that can be used in, for example, estimation processing.

Note that in the example of the training data DT4 described in FIG. 12 and the like, a case was described in which a type corresponding to a whole particle, a type corresponding to a hollow particle, a type corresponding to a damaged particle, or a type corresponding to an intermediate is set in the “type information”, but there is no limitation to this. That is, the “type information” in the training data DT4 may be set to, for example, any one of a plurality of types (hereinafter also referred to as damage type) corresponding to the damage status of the viral vector particle.

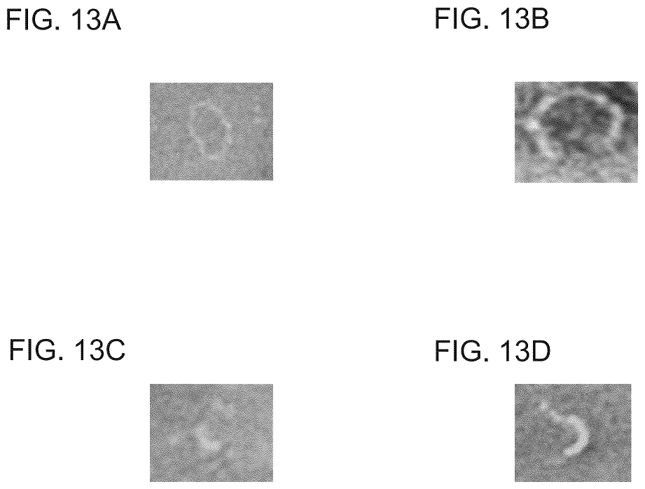

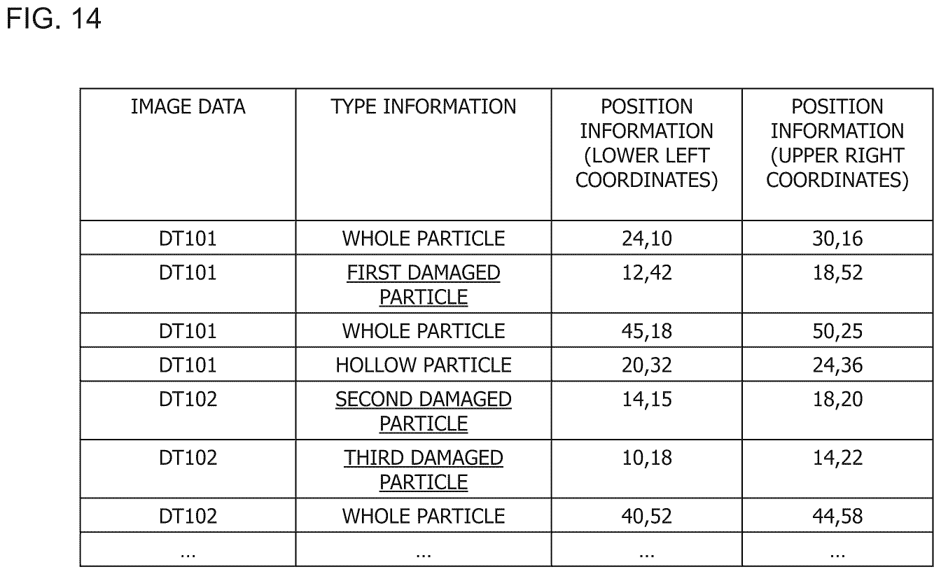

Specifically, the plurality of damage types may include, for example, a damage type indicating a viral vector particle whose shape has been deformed (hereinafter also referred to as a first damage type) as shown in FIG. 13A. Also, the plurality of damage types may include, for example, a type indicating a viral vector particle whose outer edge is at least partially damaged (hereinafter also referred to as a second damage type), as shown in FIG. 13B. In addition, the plurality of damage types may include, for example, a type indicating at least a portion of a viral vector particle that is broken into a plurality of pieces (hereinafter also referred to as a third damage type), as shown in FIGS. 13C and 13D. The training data DT4 may be set in such a manner that a plurality of damaged particles, including the first damage type, the second damage type, and the third damage type, for example, are distinguished from one another, as shown in the underlined portions of FIG. 14.

More specifically, the second damage type may be further distinguished into a plurality of damage types, for example, depending on the location of the damage on the outer edge of the particle. Specifically, the second damage type may include, for example, a type indicating a damaged particle in which a portion of the outer edge of the particle on a side in a specified direction (e.g., the upward side in the image data DT1) is damaged, and a type indicating a damaged particle in which a portion of the outer edge of the particle on a side different from the side in the specified direction (e.g., the downward side in the image data DT1) is damaged.

This makes it possible for the information processing device 1 in this modified example to further improve the detection accuracy of each viral vector particle, for example.

### Flowchart of Estimation Processing in First Embodiment

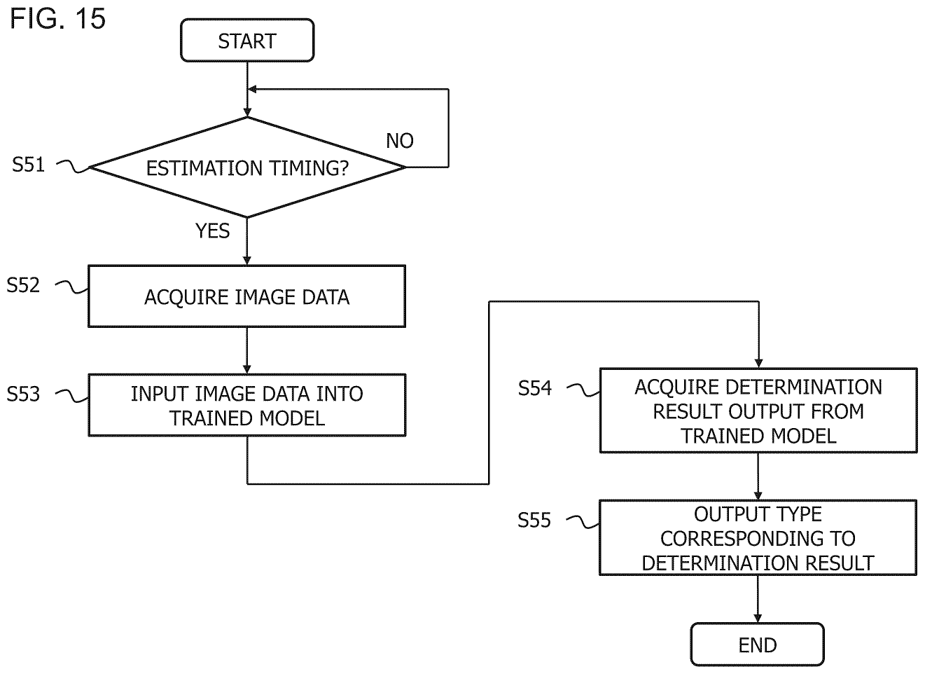

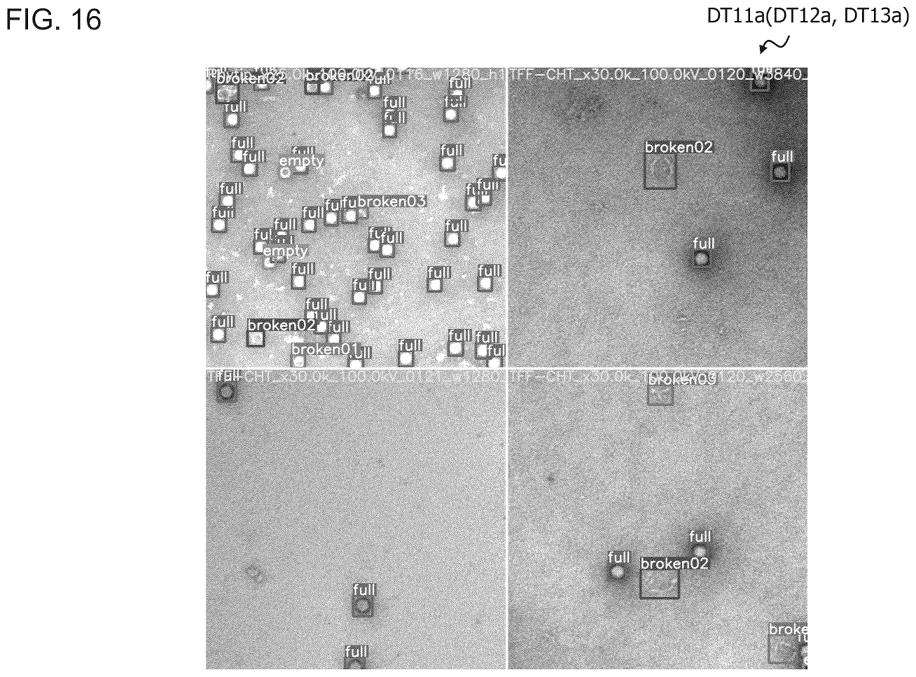

Next, the estimation processing in the first embodiment will be described. FIG. 15 is a flowchart illustrating the estimation processing according to the first embodiment. FIG. 16 is a diagram illustrating the estimation processing in the first embodiment.

As shown in FIG. 15, the image generation unit 111 waits until the estimation timing, for example (NO in step S51). The estimation timing may be, for example, a timing designated by the operator. Specifically, the estimation timing may be, for example, a timing after the trained model MD is generated in the training processing.

Then, when the estimation timing is reached (YES in step S51), the image generation unit 111 acquires image data DT11 in which, for example, a viral vector particle appears (step S52). Specifically, the image generation unit 111 acquires the image data DT11 captured by the TEM from the TEM, for example.

Next, the image input unit 115 inputs, for example, the image data DT11 acquired in the processing of step S52 into the trained model MD (step S53).

Furthermore, the result acquisition unit 116 acquires, for example, an estimation result output from the trained model MD accompanying the input of the image data DT11 in the processing of step S53 (step S54). The estimation result is, for example, an estimation result regarding the combination of the type information DT12 and the position information DT13 corresponding to the viral vector particle appearing in the image data DT11 acquired in the processing of step S52.

Thereafter, the type output unit 117 outputs, for example, the estimation result acquired in the processing of step S54 to the operation terminal 2 as a combination of the type information DT12 and the position information DT13 corresponding to the viral vector particles appearing in the image data DT11 obtained in the processing of step S52 (step S55).

Specifically, as shown in FIG. 16, the type output unit 117 displays, on the operation terminal 2, the image data DT11a associated with the combination of the type information DT12a of each viral vector particle and the position information DT13a of each viral vector particle (a bounding box including the position information DT13a), for example. In the example shown in FIG. 16, “full” corresponds to a type indicating a whole particle, “empty” corresponds to a type indicating a hollow particle, “broken01” corresponds to the first damage type, “broken02” corresponds to the second damage type, and “broken03” corresponds to the third damage type.

This allows the operator to view, for example, the estimation results of type information DT12 and position information DT13 corresponding to the viral vector particles appearing in the image data DT11 acquired in the processing of step S52.

In this manner, in the training processing, the information processing device 1 in this embodiment generates, for example, a plurality of pieces of image data DT1 (training image data DT1) in which test subject viral vector particles appear. Then, the information processing device 1 receives, for each of the generated pieces of image data DT1, input of a combination of type information DT2 of the viral vector particle appearing in the image data DT1 and position information DT3 of the viral vector particle in the image data

DT1. Next, the information processing device 1 generates a plurality of pieces of training data DT4 by, for example, associating the received combinations of the type information DT2 and the position information DT3 with the respective plurality of pieces of image data DT1. Thereafter, the information processing device 1 generates a trained model MD by, for example, performing machine learning on the generated plurality of pieces of training data DT4.

Furthermore, in the estimation processing, the information processing device 1 in this embodiment inputs, for example, image data DT11 (determination image data DT11) in which a viral vector particle appears, to the trained model MD. Then, the information processing device 1 acquires, for example, an estimation result output from the trained model MD accompanying the input of the image data DT11. Thereafter, the information processing device 1 outputs the acquired estimation result as, for example, the combination of the type information DT12 and the position information DT13 corresponding to the viral vector particle appearing in the input image data DT11.

That is, the information processing device 1 in this embodiment generates a trained model MD by using, for example, a plurality of pieces of training data DT4 including not only the image data DT1 and the type information DT2 of the viral vector particles, but also the position information DT3 of the viral vector particles in the image data DT1.

This enables the information processing device 1 in this embodiment to detect, for example, each viral vector particle without manual intervention. Also, the information processing device 1 can, for example, detect each viral vector particle with high accuracy.

### [Method for Generating Image Data DT1]

Next, a specific example of a method for generating image data DT1 (hereinafter also simply referred to as an image generation method) in the first embodiment will be described. Below, a case will be described in which the viral vector particle is an AAV vector particle.

The image generation method includes, for example, preparing AAV vector particles to be imaged by TEM. Specifically, the preparation of AAV vector particles includes, for example, an operation of preparing AAV vector particles by cell culture (hereinafter also referred to as operation a), an operation of concentrating the culture supernatant by ultrafiltration (hereinafter also referred to as operation b), and an operation of affinity purifying the AAV vector particles after operation b (hereinafter also referred to as operation c). Specifically, the amount of AAV vector particles subjected to ultrafiltration in operation b may be, for example, about 10 to the power of 12 (about 10 mL). Also, the amount of AAV vector particles subjected to affinity purification in step c may be, for example, about 10 to the power of 12 (about 5 mL).

The image generation method includes, for example, an operation of washing the AAV vector particles after operation c is performed (hereinafter also referred to as operation d), and an operation of staining the AAV vector particles after operation d is performed, with a predetermined staining agent (hereinafter also referred to as operation e).

Specifically, operation d may include, for example, an operation of placing 3 μL of the AAV vector particle solution on a support membrane (grid) and leaving it for 60 seconds (hereinafter also referred to as operation d1), an operation of removing moisture by vertically placing filter paper against the side of the membrane on which the AAV vector particles are placed after operation d1 has been performed (hereinafter also referred to as operation d2), and an operation of adding 3 μL of water to the AAV vector particles after operation d2 has been performed and leaving it for 10 seconds (hereinafter also referred to as operation d3). Furthermore, each of the operations d2 and d3 may be performed a plurality of times (e.g., three times) in this order. Note that an example of a commercially available support membrane used in operation d1 is “Collodion Membrane Attached Mesh” manufactured by Nissin EM Co., Ltd.

In addition, the operation e may include, for example, an operation of placing a predetermined staining agent on the membrane on which the AAV vector particles are placed after operation d has been performed and leaving it for 60 seconds (hereinafter also referred to as operation e1), an operation of removing moisture by vertically placing filter paper against the side of the membrane on which the AAV vector particles are placed after the operation e1 has been performed (hereinafter also referred to as operation e2), and an operation of placing the membrane on which the AAV vector particles are placed on the filter paper with the side on which the AAV vector particles are placed facing downward and leaving it for 120 seconds after operation e2 has been performed (hereinafter also referred to as operation e3).

Thereafter, in the image generation method, for example, image data DT1 is generated by capturing an image of the AAV vector particle after operation e has been performed using the TEM.

That is, in the image generation method of this embodiment, by performing the operation b, it is possible to prepare each piece of image data DT1 so that a sufficient number of AAV vector particles (e.g., approximately 300to 400 particles) appear in the image data. Furthermore, in the image generation method, by performing operations c, d, and e, it is possible to increase the clarity of the AAV vector particles appearing in the image data DT1, for example.

This makes it possible for the image generation method to generate image data DT1 suitable for generating a trained model MD, for example. For this reason, in the training processing in this embodiment, for example, it is possible to generate a trained model MD with high determination accuracy.

Note that the predetermined staining agent used in operation e may be, for example, a negative staining reagent containing phosphotungstic acid, ammonium molybdate, methylamine tungstate or methylamine vanadate as an active ingredient, and a negative staining reagent containing methylamine tungstate or methylamine vanadate as an active ingredient is preferred. This makes it possible for the image generation method to, for example, further increase the clarity of AAV vector particles appearing in the image data DT1.

In addition, in the image generation method, for example, not all of the operations b, c, d, and e need to be performed. That is, in the image generation method, for example, some of the operations b, c, d, and e may not be performed.

### REFERENCE SIGNS LIST

1: INFORMATION PROCESSING DEVICE
2: OPERATION TERMINAL
10: INFORMATION PROCESSING SYSTEM
101: CPU
102: MEMORY
103: COMMUNICATION INTERFACE
104: STORAGE MEDIUM
105: BUS
110: PROGRAM
111: IMAGE GENERATION UNIT
112: TYPE INPUT RECEPTION UNIT
113: TRAINING DATA GENERATION UNIT
114: TRAINED MODEL GENERATION UNIT
115: IMAGE INPUT UNIT
116: RESULT ACQUISITION UNIT
117: TYPE OUTPUT UNIT
130: STORAGE UNIT
DT1: IMAGE DATA
DT2: TYPE INFORMATION
DT3: POSITION INFORMATION
DT4: TRAINING DATA
DT11: IMAGE DATA
DT12: TYPE INFORMATION
DT13: POSITION INFORMATION
MD: TRAINED MODEL

1. A viral particle detection method for detecting a type of a viral particle that is a test subject, comprising:
generating a plurality of pieces of training image data in which the viral particle appears;
receiving, for each of the generated plurality of pieces of training image data, input of a combination of a type of the viral particle appearing in the training image data and position information of the viral particle in the training image data;
generating a plurality of pieces of training data by associating the combinations input for each of the plurality of pieces of training image data with the respective pieces of training image data;
generating a trained model by performing machine learning on the generated plurality of pieces of training data;
inputting a determination image data in which the viral particle appears into the trained model;
acquiring an estimation result output from the trained model accompanying input of the determination image data;
outputting the acquired estimation result as the combination corresponding to the viral particle appearing in the determination image data.

2. The viral particle detection method according to claim 1,
wherein in the step of receiving input, input of any one of a plurality of types including a type indicating a whole particle, a type indicating a hollow particle, a type indicating a damaged particle, and a type indicating an intermediate is received as the type of the viral particle appearing in each of the plurality of pieces of training image data.

3. The viral particle detection method according to claim 2,
wherein the type indicating the damaged particle includes a plurality of damage types corresponding to a damage status of the viral particle, and
in the step of receiving input, input of any one of the plurality of types including the plurality of damage types is received as the type of the viral particle appearing in each of the plurality of pieces of training image data.

4. The viral particle detection method according to claim 3,
wherein the plurality of damage types include a type indicating the viral particle whose shape is deformed, a type indicating the viral particle whose outer edge is at least partially damaged, and a type indicating the viral particle that is broken into a plurality of pieces.

5. The viral particle detection method according to claim 3,
wherein the plurality of damage types include a type indicating the viral particle whose outer edge on a side in a predetermined direction is partially damaged, and a type indicating the viral particle whose outer edge on a side in a direction different from the predetermined direction is partially damaged.

6. The viral particle detection method according to claim 1, further comprising:
culturing the viral particle before the step of generating the training image data;
subjecting the cultured viral particle to ultrafiltration;
subjecting the ultrafiltered viral particle to affinity purification;
washing the viral particle subjected to affinity purification;
staining the washed viral particle with a predetermined staining agent; and
generating the plurality of pieces of training image data in which the stained viral particle appears, in the step of generating the plurality of pieces of training image data.

7. The viral particle detection method according to claim 6,
wherein the predetermined staining agent is a negative staining reagent containing methylamine tungstate or methylamine vanadate as an active ingredient.

8. An information processing device for detecting a type of a viral particle that is a test subject, comprising:
an image generation unit configured to generate a plurality of pieces of training image data in which the viral particle appears;
a type input reception unit configured to, for each of the generated plurality of pieces of training image data, receive input of a combination of the type of the viral particle appearing in the training image data and position information of the viral particle in the training image data;
a training data generation unit configured to generate a plurality of pieces of training data by associating the combinations input for each of the plurality of pieces of training image data with the respective plurality of pieces of training image data;
a trained model generation unit configured to generate a trained model by performing machine learning on the generated plurality of pieces of training data;
an image input unit configured to input determination image data in which the viral particle appears into the trained model;
a result acquisition unit configured to acquire an estimation result output from the trained model accompanying input of the determination image data; and
a type output unit configured to output the acquired estimation result as the combination corresponding to the viral particle appearing in the determination image data.

9. A viral particle detection program for causing a computer to execute processing for detecting a type of a viral particle that is a test subject, the viral particle detection program comprising:
generating a plurality of pieces of training image data in which the viral particle appears;
receiving, for each of the generated plurality of pieces of training image data, input of a combination of a type of the viral particle appearing in the training image data and position information of the viral particle in the training image data;
generating a plurality of pieces of training data by associating the combinations input for each of the plurality of pieces of training image data with the respective plurality of pieces of training image data;
generating a trained model by performing machine learning on the generated plurality of pieces of training data;
inputting determination image data in which the viral particle appears into the trained model;
acquiring an estimation result output from the trained model accompanying input of the determination image data; and
outputting the acquired estimation result as the combination corresponding to the viral particle appearing in the determination image data.
